(19) United States
(12) Patent Application Publication
(10) Pub. No.: US 2017/0100549 A1
(43) Pub. Date: Apr. 13, 2017

(54) COMPOSITE ANESTHESIA DEVICE FOR MEDICAL USE

(71) Applicant: Hak Beom KIM, Gyeonggi-do (KR)

(72) Inventor: Hak Beom KIM, Gyeonggi-do (KR)

(21) Appl. No.: 15/128,040

(22) PCT Filed: Sep. 19, 2014
(86) PCT No.: PCT/KR2014/008744

§ 371 Date: Sep. 21, 2016

(30) Foreign Application Priority Data
Mar. 28, 2014 (KR) 20-2014-0002491

(51) Int. Cl. A61M 11/00; A61M 19/00; A61M 5/20; A61M 5/24

(52) U.S. Cl. CPC A61M 11/006; A61M 5/24; A61M 19/00; A61M 5/20; A61M 2202/048

(57) ABSTRACT

The present invention relates to a composite anesthesia device for medical use, which allows a syringe for injecting local anesthesia into the body through a needle to be added with a function for spraying a topical anesthetic so as to anesthetize the skin tissue of a corresponding portion through the surface anesthesia spray before the needle is inserted into the body, thereby reducing the pain generated when injecting the anesthetic.

### FIELD

The present invention generally relates to a composite anesthesia device for medical use. More particularly, the present invention relates to a composite anesthesia device for medical use, the device allowing a syringe for injecting a local anesthetic into the body through a needle to be added with a function for spraying a topical anesthetic so as to anesthetize the skin tissue of a corresponding portion through the topical anesthetic spray before the needle is inserted into the body, thereby reducing the pain generated when injecting the anesthetic.

### BACKGROUND ART

In general, local anesthesia is a technique to anesthetize only a target area by directly injecting an anesthetic, such as lidocaine, into the target area. Local anesthesia is used in aesthetic surgery on eyes or noses or in dental procedures, such as a dental implant or a tooth extraction. Further, local anesthesia is widely used in dermatology, orthopedics, and surgery depending on a purpose of an operation or a surgical procedure.

Here, local anesthesia is produced not by only one injection of the anesthetic but by several injections of the anesthetic depending on an area of an operation or a surgical procedure. Further, in the case of a target area containing a high density of sensory nerve endings, patients feel severe pain whenever the target area is pierced with a needle for injecting anesthetic.

Meanwhile, a local anesthetic spray apparatus for anesthetizing or desensitizing a specific part of the skin has been disclosed. However, the local anesthetic spray apparatus is problematic in that it should be provided separated from the conventional local anesthetic syringe, and the use thereof is inconvenient because local anesthesia is not produced simultaneously. The local anesthetic spray apparatus is further problematic in that the size thereof is big, so when the sophisticated surgical procedure is required to be performed, it is difficult to use the local anesthetic spray.

### Technical Problem

Accordingly, the present invention has been made keeping in mind the above problems occurring in the related art, and the present invention is intended to propose a composite anesthesia device for medical use, the device allowing a syringe for injecting a local anesthetic into the body through a needle to be added with a function for spraying a topical anesthetic so as to anesthetize or desensitize the skin tissue of a corresponding portion through the topical anesthetic spray before the needle is inserted into the body, thereby reducing the pain generated when injecting the anesthetic.

The present invention is further intended to propose a composite anesthesia device for medical use, whereby the device can be easily used in a sophisticated operation or surgical procedure, and allows a topical anesthetic to be accurately sprayed to a specific skin tissue that is required to achieve local anesthesia.

### Technical Solution

In order to achieve the above object, according to one aspect of the present invention, there is provided a composite anesthesia device for medical use, the anesthesia device including: a main body including: a cartridge replaceably mounted therein, the cartridge being charged with an anesthetic; a needle provided at a front of the main body and connected to the cartridge so as to supply the anesthetic; and a controller provided on a first side of the main body, and controlling an injection amount of the anesthetic through the needle by manipulation thereof; a coupling unit provided on a second side of the main body to be in parallel to the needle, and including a spray nozzle, an adapter, and a control knob respectively provided at a front, at a back, and on a side thereof; and a topical anesthetic cylinder detachably connected to the adapter with a topical anesthetic being charged therein, and configured such that the topical anesthetic is sprayed through the spray nozzle by manipulation of the control knob.

The main body may be in a form of a manual syringe, a syringe gun, or an electric syringe, and an injection type is selected between a manual type or an electric type.

The spray nozzle may further include: a sub-spray nozzle provided in the spray nozzle to be extendable in a forward direction; and a channel provided on a side of the spray nozzle, with a control protrusion being formed by extending from the sub-spray nozzle and being protrudingly disposed in the channel, wherein the sub-spray nozzle is controlled to be drawn in and out of the spray nozzle in response to forward and backward movements of the control protrusion in the channel.

### Advantageous Effects

According to the present invention having the above-described characteristics, it is possible to reduce the pain generated when injecting the anesthetic by allowing a syringe for injecting a local anesthetic into the body through a needle to be added with a function for spraying a topical anesthetic so as to anesthetize the skin tissue of a corresponding portion through a topical anesthetic spray before the needle is inserted into the body.

### DESCRIPTION OF DRAWINGS

FIG. 1 is a perspective view showing a composite anesthesia device for medical use according to an embodiment of the present invention;

FIG. 2 is an operational view showing the composite anesthesia device for medical use according to the embodiment of the present invention;

FIG. 3 is an operational view showing a modification of FIG. 1;

FIG. 4 is a perspective view showing a composite anesthesia device for medical use according to another embodiment of the present invention;

FIG. 5 is an operational view showing a modification of FIG. 4; and

FIG. 6 is a perspective view showing a composite anesthesia device for medical use according to a further embodiment of the present invention.

### MODE FOR INVENTION

Reference will now be made in greater detail to exemplary embodiments of the present invention, an example of which is illustrated in the accompanying drawings. Further, the embodiments of the present invention are disclosed only for illustrative purposes and should not be construed as limiting the present invention.

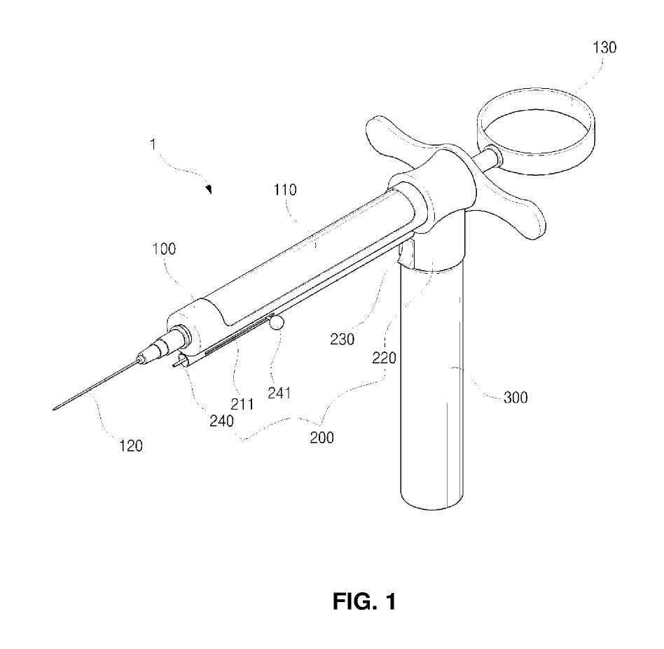

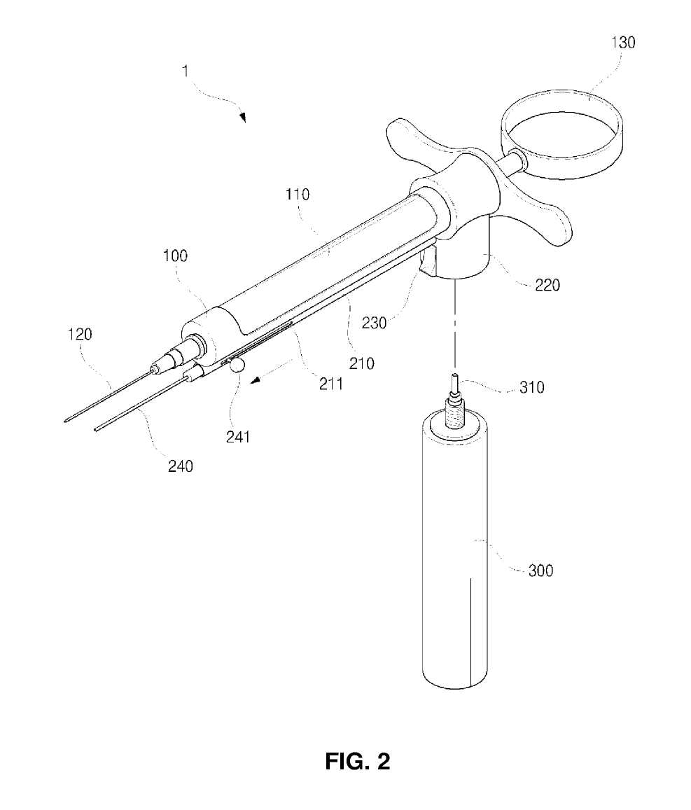

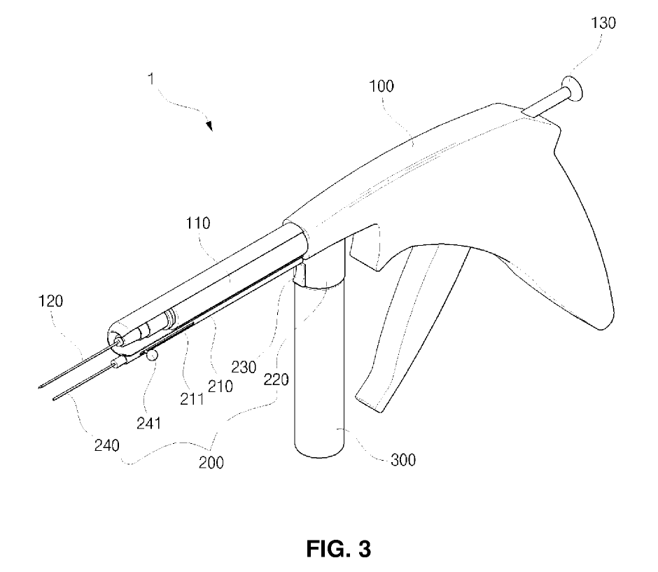

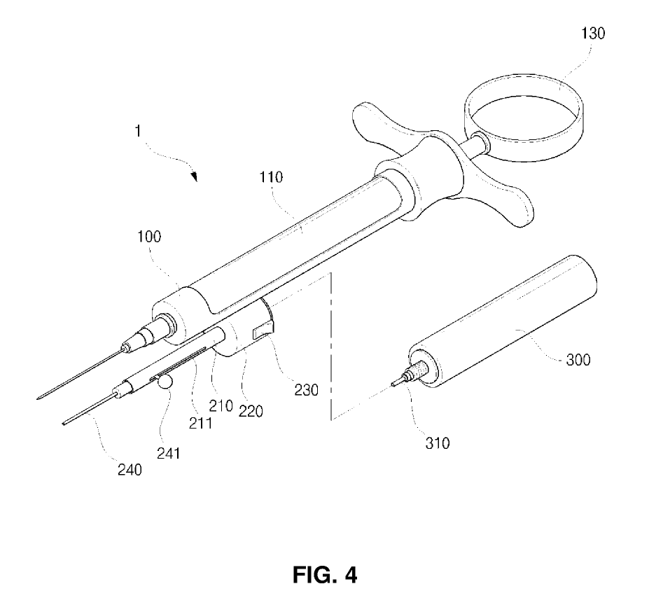

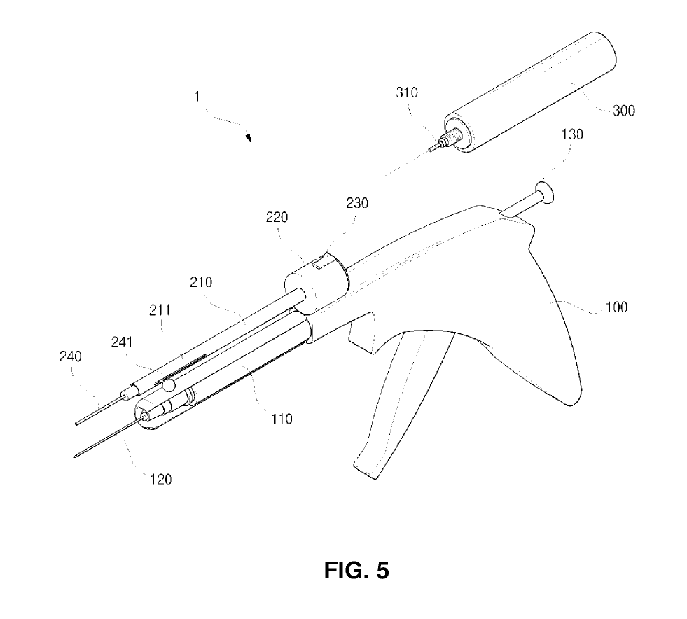

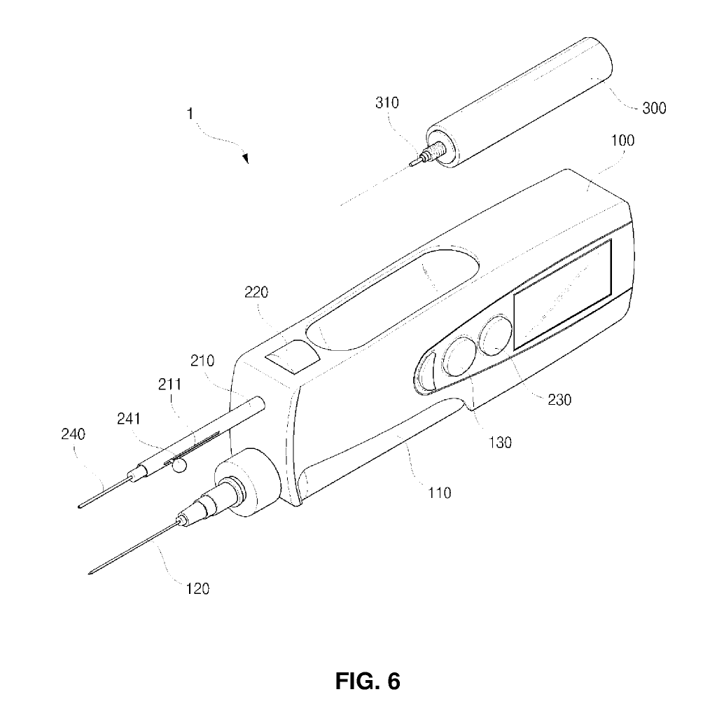

FIG. 1 is a perspective view showing a composite anesthesia device for medical use according to an embodiment of the present invention; FIG. 2 is an operational view showing the composite anesthesia device for medical use according to the embodiment of the present invention; FIG. 3 is an operational view showing a modification of FIG. 1; FIG. 4 is a perspective view showing a composite anesthesia device for medical use according to another embodiment of the present invention; FIG. 5 is an operational view showing a modification of FIG. 4; and FIG. 6 is a perspective view showing a composite anesthesia device for medical use according to a further embodiment of the present invention.

A composite anesthesia device for medical use according to a preferred embodiment of the present invention includes: a main body 100 including: a cartridge 110 replaceably mounted therein, the cartridge being charged with an anesthetic; a needle 120 provided at a front of the main body and connected to the cartridge 110 so as to supply the anesthetic; and a controller 130 provided on a first side of the main body, and controlling an injection amount of the anesthetic through the needle 120 by manipulation thereof; a coupling unit 200 provided on a second side of the main body 100 to be in parallel to the needle 120, and including a spray nozzle 210, an adapter 220, and a control knob 230 respectively provided at a front, at a back, and on a side thereof; and a topical anesthetic cylinder 300 detachably connected to the adapter 220 with a topical anesthetic being charged therein, and configured such that the topical anesthetic is sprayed through the spray nozzle 210 by manipulation of the control knob 230.

Reference will be made in greater detail to elements and connections of the composite anesthesia device for medical use 1 according to the preferred embodiment of the present invention with reference to FIGS. 1 to 6, hereinbelow.

Herein, the main body 100 serves to allow the anesthetic to be injected into a body and allow elements that will be described hereinafter to be mounted thereto so as to be operated. The main body 100 includes: the cartridge 110 replaceably mounted therein, the cartridge being charged with the anesthetic (preferably, a lidocaine ample); the needle 120 provided at the front of the main body and connected to the cartridge 110 so as to supply the anesthetic; and the controller 130 provided on the first side of the main body, and controlling the injection amount of the anesthetic through the needle 120 by manipulation thereof.

The main body 100 may be in the form of a manual syringe as shown in FIGS. 1 to 3, in the form of a syringe gun as shown in FIGS. 4 and 5, or in the form of an electric syringe as shown in FIG. 6. Further, injection of the anesthetic is controlled by the controller 130. Here, a method for controlling the controller 130 may be configured to manually inject fluid into a body by a practitioner pressing the controller 130 by hand, or may be configured to automatically inject fluid by pressing the controller 130 using a predetermined electric apparatus after a practitioner sets an injection amount.

For simplicity of description, detailed description of operation of the controller 130 of the main body 100 and use of the cartridge 110 and the needle 120 is omitted since operation of the controller and use of the cartridge and the needle are identical to a manual syringe, a syringe gun, and an electric syringe for anesthesia according to the related art, so they can be inferred from the related art.

Meanwhile, the coupling unit 200 is provided under the needle 120 of the main body 100, wherein the coupling unit 200 is coupled to a topical anesthetic cylinder 300 such that the charged gas is sprayed and the coupling unit serves to allow the topical anesthetic to be sprayed from the coupled topical anesthetic cylinder 300 to the target area. The coupling unit 200 is disposed to be in parallel to the needle 120, and provided with a spray nozzle 210, an adapter 220, and a control knob 230 respectively at a front, at a back, and on a side thereof.

The spray nozzle 210 is provided right under the needle 120 to be in parallel to the needle 120, which allows the anesthetic to be injected right after the topical anesthetic is sprayed. Further, the spray nozzle 210 is further provided a sub-spray nozzle 240 therein to be extendable in a forward direction, and thereby it is possible to accurately spray the topical anesthetic on the target area that is required to achieve local anesthesia, by being disposed close to the target area.

Here, the spray nozzle 210 may include a channel 211 provided on a side of the spray nozzle 210, with a control protrusion 241 being formed by extending from the sub-spray nozzle 240 and being protrudingly disposed in the channel 211, wherein the sub-spray nozzle 240 is controlled to be drawn in and out of the spray nozzle 210 in response to forward and backward movements of the control protrusion 241 in the channel 211. Further, the adapter 220 is engaged with the topical anesthetic cylinder 300, wherein as soon as the adapter is engaged with the topical anesthetic cylinder, the topical anesthetic charged in the cylinder is ready to be sprayed out through the front end of the spray nozzle 210. Further, the control knob 230 serves to control spraying the topical anesthetic ready to be sprayed.

Further, as shown in FIGS. 2 and 4, the adapter 220 may be disposed to allow the topical anesthetic cylinder 300 to be vertically coupled thereto, or as shown in FIGS. 3 and 5, may be disposed to allow the topical anesthetic cylinder to be horizontally coupled thereto.

For simplicity of description, detailed description of how to spray the topical anesthetic through the adapter 220 and the control knob 230 is omitted since it is identical to a method for spraying and controlling gas according to the related art, so it can be inferred from the related art.

Meanwhile, the adapter 220 is coupled with the topical anesthetic cylinder 300 to allow the topical anesthetic to be supplied to the adapter, wherein the topical anesthetic is charged in the topical anesthetic cylinder 300 so as to anesthetize a skin tissue or desensitize sensation when sprayed. Herein, the topical anesthetic is sprayed through the spray nozzle 210 by manipulation of the control knob 230.

The topical anesthetic cylinder 300 is engaged with at a side of the adapter 220, wherein the topical anesthetic cylinder is protrudingly provided with a supply pipe 310 at a location with which the adapter is engaged, so the topical anesthetic cylinder 300 is connected to the coupling unit 200 to allow the topical anesthetic to be supplied to the coupling unit by connecting the supply pipe 310 with the adapter 220. In other words, as soon as the supply pipe 310 is engaged with the adapter 220, the topical anesthetic in the topical anesthetic cylinder is compressed and ready to be supplied, and the spray of the topical anesthetic is determined by using the control knob 230.

Further, when the charged topical anesthetic is used up, the topical anesthetic cylinder 300 may be reused by recharging the charged topical anesthetic, and the volume of the reusable topical anesthetic cylinder 300 may be changed depending on the size of the main body 100. Further, the topical anesthetic cylinder 300 may be engaged with the coupling unit 200 as in the preferred embodiment of the present invention, but is not limited thereto.

Although a preferred embodiment of the present invention has been described for illustrative purposes, those skilled in the art will appreciate that various modifications, additions and substitutions are possible, without departing from the scope and spirit of the invention as disclosed in the accompanying claims.

### DESCRIPTION OF REFERENCE CHARACTERS OF IMPORTANT PARTS

1: composite anesthesia device for medical use

100: main body 110: cartridge

120: needle 130: controller

200: coupling unit 210: spray nozzle

211: channel 220: adapter

230: control knob 240: sub-spray nozzle

241: control protrusion 300: topical anesthetic cylinder

310: supply pipe

1-3. (canceled)

4. A composite anesthesia device, comprising:
a main body including:
a cartridge replaceably mounted therein, the cartridge being charged with an anesthetic;
a needle provided at a front of the main body and connected to the cartridge; and
a controller provided on a first side of the main body, and controlling an injection amount of the anesthetic through the needle by manipulation thereof;
a coupling unit provided on a second side of the main body to be in parallel to the needle, and including a spray nozzle, an adapter, and a control knob respectively provided at a front, at a back, and on a side thereof; and
a topical anesthetic cylinder detachably connected to the adapter with a topical anesthetic being charged therein, and configured such that the topical anesthetic is sprayed through the spray nozzle by manipulation of the control knob.

5. The composite anesthesia device of claim 4, wherein the main body is in a form of a manual syringe, a syringe gun, or an electric syringe.

6. The composite anesthesia device of claim 4, wherein an injection type is selected between a manual type or an electric type.

7. The composite anesthesia device of claim 4, wherein the spray nozzle further includes: a sub-spray nozzle provided in the spray nozzle to be extendable in a forward direction; and a channel provided on a side of the spray nozzle, with a control protrusion being formed by extending from the sub-spray nozzle and being protrudingly disposed in the channel, wherein the sub-spray nozzle is controlled to be drawn in and out a of the spray nozzle in response to forward and backward movements of the control protrusion in the channel.

8. The composite anesthesia device of claim 5, wherein
the spray nozzle further includes: a sub-spray nozzle provided in the spray nozzle to be extendable in a forward direction; and a channel provided on a side of the spray nozzle, with a control protrusion being formed by extending from the sub-spray nozzle and being protrudingly disposed in the channel, wherein the sub-spray nozzle is controlled to be drawn in and out a of the spray nozzle in response to forward and backward movements of the control protrusion in the channel.
